(19) United States
(12) Patent Application Publication
(10) Pub. No.: US 2019/0194701 A1
(43) Pub. Date: Jun. 27, 2019

(54) METHOD FOR PRODUCING BUTYRIC ACID AND/OR ITS SALTS

(71) Applicant: GREEN CELLULOSITY CORPORATION, Hsinchu City (TW)

(72) Inventors: Chiang-Hsiung TONG, Hsinchu City (TW); Chun-Han CHEN, Hsinchu City (TW); Wan-Shan CHIEN, Hsinchu City (TW); Ruey-Fu SHIH, Hsinchu City (TW); Jheng-Jin LUO, Hsinchu City (TW)

(21) Appl. No.: 16/222,547

(22) Filed: Dec. 17, 2018

Related U.S. Application Data
(60) Provisional application No. 62/609,170, filed on Dec. 21, 2017.

(30) Foreign Application Priority Data
Jun. 12, 2018 (TW) 107120222
Sep. 7, 2018 (TW) 107131552

(51) Int. Cl. C12P 7/52; C12N 1/20; C12P 39/00; C12P 7/16; C12R 1/145; C12R 1/245

(52) U.S. Cl. CPC C12P 7/52; C12N 1/20; C12R 1/245; C12P 7/16; C12R 1/145; C12P 39/00

(57) ABSTRACT

A method for producing butyric acid and/or a butyrate is provided, wherein the method comprises fermenting a saccharide-containing substrate in the presence of a first strain and a second strain, wherein the first strain is a butyric acid bacterium and the second strain is at least one of a homofermentative lactic acid bacterium and a facultative heterofermentative lactic acid bacterium.

### CROSS REFERENCE TO RELATED APPLICATIONS

This application claims priority to U.S. Provisional Application Ser. No. 62/609,170 filed on Dec. 21, 2017, in the United States Patent and Trademark Office, and to Taiwan Patent Applications No. 107120222 filed on Jun. 12, 2018 and No. 107131552 filed on Sep. 7, 2018, in the Taiwan Intellectual Property Office, the disclosures of which are incorporated herein in their entirety by reference.

### FIELD OF THE INVENTION

The present invention relates to a method for producing butyric acid and/or a butyrate, comprising fermenting a saccharide-containing substrate in the presence of a first strain and a second strain, wherein the first strain is a butyric acid bacterium and the second strain is at least one of a homofermentative lactic acid bacterium and a facultative heterofermentative lactic acid bacterium. As compared to the conventional method for producing butyric acid, the method of the present invention can increase the yield of butyric acid and decrease the production of by-product.

### BACKGROUND OF THE INVENTION

Butyric acid and its derivatives have been wildly used in industry and can be used as a chemical material or a food additive. It was revealed by researches that butyric acid is effective in maintaining and promoting the function of animal intestinal tract, and inducing the apoptosis of tumor cells. Therefore, butyric acid has good potential in treating gastroenteritis, cancer and leukemia. The methods for producing butyric acid including chemosynthesis and microbial catalysis, wherein the chemosynthesis is primarily the fossil oil-based process such as butyraldehyde oxidation and propylene carbonylation. In the past, chemosynthesis was the primary process for producing butyric acid because it was simple and low cost. However, the energy crisis had posed challenges, including the increased cost, to such fossil oil-based chemosynthesis process. Along with the development of applying butyric acid and its derivatives in the biological related field, the biologically derived butyric acid products are much more popular with consumers than those produced by chemosynthesis. Hence, people are paying more and more attention to the microbial catalysis method for producing butyric acid.

The microbial catalysis method for producing butyric acid is an anaerobic fermentation process, which is usually conducted in the presence of a single strain. The microorganisms suitable for such method include Clostridium sp., Butyrivibrio sp., Butyribacterium sp., Sarcina sp., Eubacterium sp., Fusobacterium sp., Megasphaera sp., etc. However, since the single-strain microbial catalysis method for producing butyric acid is deficient in such as a high material cost, a low yield per unit volume, and a high yield of by-product (i.e., acetic acid), people in this field still endeavor to develop a microbial catalysis method that is effective in increasing the yield of butyric acid and decreasing the production of by-product.

Inventors of the present invention discovered that as compared to the conventional method using microbial catalysis in the presence of a single strain, the method comprising fermenting a saccharide-containing substrate in the presence of a butyric acid bacterium and at least one of the following lactic acid bacteria can provide a better yield of butyric acid and a decreased production of by-product: a homofermentative lactic acid bacterium and a facultative heterofermentative lactic acid bacterium.

### SUMMARY OF THE INVENTION

An objective of the present invention is to provide a method for producing butyric acid and/or a butyrate, comprising fermenting a saccharide-containing substrate in the presence of a first strain and a second strain, wherein the first strain is a butyric acid bacterium and the second strain is at least one of a homofermentative lactic acid bacterium and a facultative heterofermentative lactic acid bacterium.

Preferably, the first strain is Clostridium sp. For example, the first strain is Clostridium tyrobutyricum, especially at least one of Clostridium tyrobutyricum DSM 27751 and Clostridium tyrobutyricum ATCC 25755.

Preferably, the second strain is as least one of Lactobacillus sp., Lactococcus sp., Sporolactobacillus sp., and Bacillus sp. For example, the second strain is as least one of Lactobacillus casei, Lactobacillus rhamnosus, Lactobacillus delbrueckii, Lactobacillus plantarum, Lactobacillus paracasei, Lactobacillus bulgaricusi, Lactococcus lactis, and Sporolactobacillus inulinus.

In some embodiments of the present invention, the first strain is Clostridium tyrobutyricum DSM 27751, and the second strain is as least one of Lactobacillus casei, Lactobacillus rhamnosus, Lactobacillus delbrueckii, Lactobacillus plantarum, Lactobacillus paracasei, Lactobacillus bulgaricusi, Lactococcus lactis, Bacillus coagulans, and Sporolactobacillus inulinus.

In the method in accordance with the present invention described above, the saccharide contained in the adopted substrate is at least one of glucose, fructose, lactose, sucrose, molasses, and cellobiose. Preferably, the substrate further contains a carbon source, a nitrogen source, and/or a mineral element. More preferably, the carbon source is at least one of acetic acid and acetate, and the mineral element is at least one of phosphorus, sulfur, potassium, magnesium, iron, and manganese.

The detailed technology and preferred embodiments implemented for the present invention are described in the following paragraphs for people skilled in this field to well appreciate the features of the claimed invention.

### DESCRIPTION OF THE PREFERRED EMBODIMENTS

The following will describe some of the embodiments of the present invention in detail. However, without departing from the spirit of the present invention, the present invention may be embodied in various embodiments and should not be limited to the embodiments described in the specification or defined in the appended claims.

Unless otherwise indicated herein, the expressions “a,” “an,” “the,” or the like recited in the specification of the present invention (especially in the claims) are intended to include both the singular and plural forms. The term “microorganism” includes the wild type present in nature and mutant type induced by any factors (e.g., natural factor or artificial factor). The term “fermentation” refers to a process for metabolizing one or more substrates by a microorganism in an aerobic atmosphere to produce an organic compound. The term “medium” refers to a composition providing nutrients and conditions (e.g., pH value, humidity, etc.) essential to the growth and replication of a microorganism. In general, the composition of the medium would be adjusted in accordance with the strain type of the microorganism to be incubated. For instance, adjustment onto the medium could be made by adding one or more of HCl, Ca(OH)₂, NaOH, NH₄OH, (NH₄)₂SO₄, NH₄Cl, CH₃COONH₄, K₂HPO₄, KH₂PO₄, NaH₂PO₃, Na₂HPO₃, citric acid, MgSO₄.7H₂O, FeSO₄.7H₂O, or MnSO₄.7H₂O so as to provide a medium with a desired pH value and/or desired physiochemical or physiological properties. The term “substrate” refers to a material that can be utilized during the fermentation of a microorganism, and thus, enters the metabolic pathway of the fermentation and then converts into other substance(s).

The term “butyric acid bacterium” refers to a microorganism that is capable of metabolizing saccharide into butyric acid during the fermentation. The term “lactic acid bacterium” refers to a microorganism that is capable of metabolizing saccharide into lactic acid during the fermentation. Depending on the metabolization characters, the lactic acid bacteria can be classified into three classes: homofermentative lactic acid bacterium, facultative heterofermentative lactic acid bacterium, and heterofermentative lactic acid bacterium. The term “homofermentative lactic acid bacterium” refers to a lactic acid bacterium that metabolizes most of the saccharide into lactic acid. The term “heterofermentative lactic acid bacterium” refers to a lactic acid bacterium that metabolizes some of the saccharide into lactic acid and some into ethanol, acetic acid and carbon dioxide. Depending on the fermentation condition (e.g., the adopted substrate, pH value, temperature, etc.), the “facultative heterofermentative lactic acid bacterium” can conduct the homo-fermentation or hetero-fermentation.

In this specification, the term “yield of butyric acid” refers to the ratio between the amount of the produced butyric acid (gram) and the total amount of the consumed saccharide (gram) and/or lactic acid (gram) during the fermentation, and is calculated by Formula 1 as follows:

$$yield of butyric acid = \frac{produced butyric acid ( gram )}{\begin{matrix}consumed saccharide ( gram ) + \\ consumed lactic acid ( gram )\end{matrix}} \qquad Formula 1$$

In this specification, the term “yield of lactic acid” refers to the ratio between the amount of the produced lactic acid (gram) and the amount of the consumed saccharide (gram) during the fermentation, and is calculated by Formula 2 as follows:

$$yield of lactic acid = \frac{produced la tic acid ( gram )}{consumed saccharide ( gram )} \qquad Formula 2$$

Different from the prior art's method of producing butyric acid by using a single butyric acid bacterium, the present invention provides a method for producing butyric acid and/or a butyrate, which comprises fermenting a saccharide-containing substrate in the presence of a first strain and a second strain, wherein the first strain is a butyric acid bacterium and the second strain is at least one of a homofermentative lactic acid bacterium and a facultative heterofermentative lactic acid bacterium. As shown in the appended Examples, as compared to the prior art, the method of the present invention can provide a better yield of butyric acid and a decreased production of by-product.

In the method in accordance of the present invention, the substrate comprises a saccharide. Examples of suitable saccharide (also called “carbohydrate”) include, but are not limited to, monosaccharides (e.g., glucose, fructose, galactose, mannose, arabinose, lyxose, ribose, ribulose, xylulose, allose, altrose, gulose, idose, talose, psicose, sorbose, tagatose); disaccharides (e.g., sucrose, maltose, lactose, lactulose, trehalose, cellobiose); oligosaccharides (e.g., stachyose, maltotriose, maltotetrose, maltopentaose); polysaccharides (e.g., starch, cellulose, glycogen, cyclodextrin, arabinoxylans, guar gum, gum arabic, chitin, gum, alginate, pectin, gellan); and molasses. In some embodiments of the present invention, a substrate containing at least one of glucose, fructose, sucrose and molasses was used to provide the desired carbon source for the fermentation.

Optionally, the substrate adopted in the method of the present invention could further contain an amino acid source. Examples of suitable amino acid sources include, but are not limited to, yeast extract, protein hydrolysate, peptone, corn steep liquor (CSL), whey, soybean meal, fish meal, meat bone meal, yeast powder, and soybean powder.

Examples of butyric acid bacterium suitable for being the first strain of the method of the present invention include, but are not limited to, Clostridium sp., Butyrivibrio sp., Butyribacterium sp., Sarcina sp., Eubacterium sp., Fusobacterium sp., and Megasphaera sp. Preferably, the first strain is Clostridium sp. More preferably, the first strain is Clostridium tyrobutyricum, such as Clostridium tyrobutyricum DSM 27751 and Clostridium tyrobutyricum ATCC 25755.

Examples of homofermentative lactic acid bacterium and facultative heterofermentative lactic acid bacterium suitable for being the second strain of the method of the present invention include, but are not limited to, Lactobacillus sp., Lactococcus sp., Sporolactobacillus sp., and Bacillus sp. Examples of Lactobacillus sp. include Lactobacillus casei, Lactobacillus rhamnosus, Lactobacillus delbrueckii, Lactobacillus plantarum, Lactobacillus paracasei, and Lactobacillus bulgaricusi. Examples of Lactococcus sp. include Lactococcus lactis. Examples of Sporolactobacillus sp. include Sporolactobacillus inulinus. Examples of Bacillus sp. include Bacillus coagulans.

In some of embodiments of the present invention, the first strain is Clostridium tyrobutyricum, and the second strain is as least one of Lactobacillus casei, Lactobacillus rhamnosus, Lactobacillus delbrueckii, Lactobacillus plantarum, Lactobacillus paracasei, Lactobacillus bulgaricusi, Lactococcus lactis, Bacillus coagulans, and Sporolactobacillus inulinus.

In addition to the above wild-type strains, the first strain can be an engineered strain obtained by a genetic engineering procedure, as long as the engineered strain is capable of metabolizing saccharide into butyric acid during the fermentation. Similarly, the second strain needed in the method of the present invention can also be an engineered strain obtained by a genetic engineering procedure, as long as the engineered strain capable of metabolizing saccharide into lactic acid during the fermentation.

In the method of producing butyric acid and/or butyrate in accordance with the present invention, the term “anaerobic atmosphere” refers to an atmosphere that contains less than 5 ppm (part per million) of oxygen, preferably less than 0.5 ppm of oxygen, and more preferably less than 0.1 ppm of oxygen. Any suitable method can be used to provide the desired anaerobic atmosphere. For example, but is not limited to, before the fermentation is conducted, an inert gas (e.g., nitrogen, carbon dioxide) is introduced into the fermentation reactor to purge the reactor, and thus, provide the desired anaerobic atmosphere; alternatively, the fermentation is conducted in an anaerobic operation box, wherein a palladium catalyst is used to catalyze the reaction of the oxygen in the box and the hydrogen in the anaerobic gas mixture to produce water, and thus, provide the desired anaerobic atmosphere.

In the method of producing butyric acid and/or butyrate in accordance with the present invention, there is no particular limitation to the order of mixing the substrate and the strains. The substrate can be added at one time or in several batches before or during the fermentation, and the strains can be supplemented optionally. For instance, the substrate can be mixed with the strains at one time before conducting the fermentation; or, the substrate can be divided into two or more batches of the same or different amounts, and then the batches are separately added into the reactor before or during the fermentation.

In the method of producing butyric acid and/or butyrate in accordance with the present invention, there is no particular limitation to the order of mixing the first and second strain. Optionally, before or during the fermentation, the substrate can be mixed with the first and second strain at one time or in several batches. For instance, the substrate can be mixed with the first strain and second strain at one time before conducting the fermentation; or, the substrate can be mixed with any one of the first and second strain before conducting the fermentation, and then the other strain is added into the fermentation reactor during the fermentation.

Optionally, before conducting the fermentation in the method of producing butyric acid and/or butyrate in accordance with the present invention, the adopted first strain and/or second strain can be pre-cultured until they grow into the log phase. Such pre-cultured strains are used to perform the method of the present invention.

In the method of producing butyric acid and/or butyrate in accordance with the present invention, the adopted substrate could further contain a carbon source, a nitrogen source and/or a mineral element. Depending on the adopted first and second strain, the carbon source could be at least one of acetic acid, acetate and saccharides (e.g., glucose, sucrose and molasses), and the mineral element could be at least one of phosphorus, sulfur, potassium, magnesium, iron and manganese, but are not limited thereby. For example, the substrate could contain potassium dihydrogen phosphate (KH₂PO₄) to provide elements such as phosphorus and potassium, and could contain magnesium chloride or magnesium sulfate heptahydrate (MgSO₄.7H₂O) to provide magnesium element, and/or could contain ferric chloride or ferrous sulfate heptahydrate (FeSO₄. 7H₂O) to provide an iron element.

The present invention will be further illustrated in detail with specific examples as follows. However, the following examples are provided only for illustrating the present invention and the scope of the present invention is not limited thereby. The scope of the present invention will be indicated in the appended claims.

### EXAMPLES

### [Source or Ingredient of Material]:

1. RCM (Reinforced Clostridial Medium): purchased from Merck (containing meat extract: 10 g/L; peptone: 10 g/L; yeast extract: 3 g/L; D (+) glucose: 5 g/L; starch: 1 g/L; NaCl: 5 g/L; sodium acetate: 3 g/L; L-cysteine hydrochloride: 0.5 g/L; agar: 0.5 g/L; pH6.8).
2. CSL (Corn Steep Liquor): purchased from Fonenfonher company.
3. MRS medium: purchased from Merck (containing peptone: 10 g/L; meat extract: 8 g/L; yeast extract: 4 g/L; D (+) glucose: 20 g/L; K₂HPO₄: 2 g/L; polysorbate 80: 1 g/L; diammonium citrate: 2 g/L; sodium acetate: 5 g/L; magnesium sulfate: 0.2 g/L; manganese sulfate: 0.04 g/L).
4. Fermentation broth A-I, each contains the following ingredients:

| | Broth | | | | | | | | |
|---|---|---|---|---|---|---|---|---|---|
| | A | B | C | D | E | F | G | H | I |
| Saccharide, | Glucose | Glucose | Glucose | Fructose | Xylose | Sucrose | Molasses | Molasses | Glucose |
| g/L | 25 | 50 | 20 | 50 | 20 | 20 | 11 | 11 | 50 |
| Yeast extract, | 10 | 10 | 10 | 10 | 10 | 10 | 10 | 10 | 10 |
| g/L | | | | | | | | | |
| Peptone, g/L | 10 | 10 | 10 | 10 | 10 | 10 | 10 | 10 | 10 |
| (NH₄)₂SO₄, | 3 | 3 | 3 | 3 | 3 | 3 | 3 | 3 | 3 |
| g/L | | | | | | | | | |
| K₂HPO₄, g/L | 3.5 | 2 | 2 | 2 | 2 | 2 | 2 | 2 | 2 |
| MgSO₄•7H₂O, | 0.8 | 0.8 | 0.8 | 0.8 | 0.8 | 0.8 | 0.8 | 0.8 | 0.8 |
| g/L | | | | | | | | | |
| FeSO₄•7H₂O, | 0.03 | 0.03 | 0.03 | 0.03 | 0.03 | 0.03 | 0.03 | 0.03 | 0.03 |
| g/L | | | | | | | | | |
| MnSO₄•H₂O, | 0.04 | 0.04 | 0.04 | 0.04 | 0.04 | 0.04 | 0.04 | 0.04 | 0.04 |
| g/L | | | | | | | | | |
| Acetic acid, | 18 | 13 | 6 | 13 | 6 | 6 | 13 | 6 | 11 |
| ml/L | | | | | | | | | |
| pH | 7 | 7 | 7 | 7 | 7 | 7 | 7 | 7 | 7 |

### [Anaerobic Atmosphere and Deoxygenated Medium]

In the following examples, an anaerobic atmosphere was provided in an air-tight container (e.g., air-tight serum bottle, centrifuge tube) by the following operations. The air-tight container and the rubber bung were covered with aluminum foil, and then sterilized under high temperature and high pressure (121° C., 1.2 atm) to exclude the interference of other microorganisms. After the sterilization was completed, the air-tight container was put in an oven to remove the residual moisture to prevent any microorganism contamination caused by the residual moisture. Thereafter, the dried air-tight container was transferred to an anaerobic operation box. After the sealing aluminum foil was slightly loosened, the palladium catalyst (purchased from Thermo Scientific, Inc., product number: BR0042) was used to catalyze the reaction of the oxygen in the air-tight container and the hydrogen in the anaerobic gas mixture to produce water and to deplete the oxygen in the air-tight container, and thus, provide an anaerobic atmosphere.

In the following examples, all the mediums were treated as follows to be deoxygenated. First of all, the medium was prepared with desired composition. The prepared medium was sterilized under high temperature and high pressure (121° C., 1.2 atm) for 20 minutes, and then transferred into an anaerobic operation box before the medium cooled down to room temperature. Thereafter, the cap of the air-tight container in which the medium was kept was slightly loosened to release the steam contained therein. Then, with the use of the palladium catalyst, the reaction of the oxygen in the air-tight container and the hydrogen in the anaerobic gas mixture was catalyzed to produce water such that deoxygenation of medium was performed. After the medium cooled down to room temperature, L-cysteine hydrochloride (0.5 g/L) was added therein to reduce the redox potential of the medium to a range suitable for microorganism such that a deoxygenated medium was provided.

### Example 1: Fermentation of a Glucose-Containing Substrate by Using a Single Butyric Acid Bacterium or a Lactic Acid Bacterium

### 1-1. Selection of Strain

To conducted a fermentation by using a single strain, one of Clostridium tyrobutyricum DSM 27751, Lactobacillus casei ATCC 393, Lactobacillus brevis ATCC 14869, Lactococcus lactis ATCC 19435, Lactobacillus rhamnosus NRRL B-445 and Lactobacillus delbrueckii ATCC 9649 was used.

### 1-2. Fermentation Experiments

A single colony of each strain provided by Example 1-1 was individually selected. Six inoculated broths were prepared by individually inoculating 4 ml of fermentation broth A with the six selected colonies, and then 1 ml sterilized water was respectively added in each of the broths. Thereafter, the inoculated broths were kept in an anaerobic incubator at 37° C. to conduct fermentation. During the fermentation, fermentation broth samples were taken from each of the broths at 0th, 31th and 95th hours, respectively. The samples were analyzed by using Agilent 1260 HPLC analysis in combination with Aminex HPX-87H (300×7.8 mm) column so as to calculate the concentrations of glucose, lactic acid, acetic acid, propionic acid and butyric acid in the fermentation broth, and the yields of butyric acid and/or lactic acid. The results are shown in Tables 1A and 1B.

TABLE 1A

| | Strain | | | | | | | | |
|---|---|---|---|---|---|---|---|---|---|
| | Clostridium | | | Lactobacillus | | | Lactobacillus | | |
| | tyrobutyricum | | | brevis | | | casei | | |
| | DSM 27751 | | | ATCC 14869 | | | ATCC 393 | | |
| | Sampling time point (hours) | | | | | | | | |
| | 0 | 31 | 95 | 0 | 31 | 95 | 0 | 31 | 95 |
| Glucose (g/L) | 20.7 | 3.0 | 0 | 20.7 | 19.2 | 13.5 | 20.7 | 11.2 | 3.0 |
| Lactic acid (g/L) | 1.2 | 0 | 0 | 1.2 | 1.6 | 3.9 | 1.2 | 9.6 | 17.1 |
| Acetic acid (g/L) | 14.1 | 12.7 | 12 | 14.1 | 14.0 | 13.8 | 14.1 | 13.7 | 13.6 |
| Propionic acid (g/L) | 0.4 | 0.4 | 0.4 | 0.4 | 0.5 | 0.5 | 0.4 | 0.5 | 0.4 |
| Butyric acid (g/L) | 0.0 | 7.4 | 8.9 | 0.0 | 0 | 0 | 0.0 | 0 | 0 |
| Yield of butyric acid (g/g) | | | 0.41 | | | 0 | | | |
| Yield of lactic acid (g/g) | | | | | | 0.38 | | | 0.9 |

TABLE 1B

| | Strain | | | | | | | | |
|---|---|---|---|---|---|---|---|---|---|
| | Lactococcus | | | Lactobacillus | | | Lactobacillus | | |
| | lactis | | | rhamnosus | | | delbrueckii | | |
| | ATCC 19435 | | | NRRL B-445 | | | ATCC 9649 | | |
| | Sampling time point (hours) | | | | | | | | |
| | 0 | 31 | 95 | 0 | 31 | 95 | 0 | 31 | 95 |
| Glucose (g/L) | 20.7 | 16.7 | 16.2 | 20.7 | 14.1 | 8.8 | 20.7 | 18.1 | 15.2 |
| Lactic acid (g/L) | 1.2 | 4.7 | 5.4 | 1.2 | 7.2 | 12.0 | 1.2 | 3.4 | 6.0 |
| Acetic acid (g/L) | 14.1 | 13.9 | 13.9 | 14.1 | 14.0 | 13.6 | 14.1 | 14.0 | 13.9 |
| Propionic acid (g/L) | 0.4 | 0.5 | 0.6 | 0.4 | 0.5 | 0.4 | 0.4 | 0.5 | 0.6 |
| Butyric acid (g/L) | 0.0 | 0 | 0 | 0.0 | 0 | 0 | 0.0 | 0 | 0 |
| Yield of butyric acid (g/g) | | | 0 | | | 0 | | | 0 |
| Yield of lactic acid (g/g) | | | 0.95 | | | 0.91 | | | 0.88 |

As shown in the Tables 1A and 1B, the use of a single butyric acid bacterium (e.g., Clostridium tyrobutyricum DSM 27751) in the fermentation of a glucose-containing substrate provided only about 0.41 of the butyric acid yield. On the other hand, if the fermentation of a glucose-containing substrate was conducted by using a single lactic acid bacterium (e.g., Lactobacillus casei ATCC 393, Lactobacillus brevis ATCC 14869, Lactococcus lactis ATCC 19435, Lactobacillus rhamnosus NRRL B-445 and Lactobacillus delbrueckii ATCC 9649), the yield of lactic acid was between 0.88-0.95.

### Example 2: Fermentation of a Glucose-Containing Substrate by Using a Butyric Acid Bacterium in Combination with a Lactic Acid Bacterium

### 2-1. Selection of Strain

Clostridium tyrobutyricum DSM 27751 was used as the first strain. One of Lactobacillus casei ATCC 393, Lactobacillus brevis ATCC 14869, Lactococcus lactis ATCC 19435, Lactobacillus rhamnosus NRRL B-445 and Lactobacillus delbrueckii ATCC 9649 was used as the second strain.

### 2-2. Pre-Culture

(a) Clostridium tyrobutyricum DSM 27751: a single colony of this strain was selected and inoculated into 5 ml RCM medium, and the inoculated medium was statically incubated in an anaerobic incubator at 37° C. for 17 hours. Thereafter, 1 ml strain liquid was taken therefrom and added into a 3 ml RCM medium to conduct a fresh incubation for 5 hours.
(b) Lactobacillus casei ATCC 393, Lactobacillus brevis ATCC 14869, Lactococcus lactis ATCC 19435, and Lactobacillus rhamnosus NRRL B-445: a single colony of each strain was selected and inoculated into 5 ml MRS medium, and the inoculated medium was statically incubated in an anaerobic incubator at 37° C. for about 28 hours.
(c) Lactobacillus delbrueckii ATCC 9649: a single colony of this strain was selected and inoculated into 5 ml MRS medium, and the inoculated medium was statically incubated in an anaerobic incubator at 37° C. for about 3 days.

### 2-3. Fermentation Experiments

Five mixtures were prepared by mixing 0.5 ml Clostridium tyrobutyricum DSM 27751 strain liquid provided by Example 2-2 with 0.5 ml each of Lactobacillus casei ATCC 393 strain liquid, Lactobacillus brevis ATCC 14869 strain liquid, Lactococcus lactis ATCC 19435 strain liquid, Lactobacillus rhamnosus NRRL B-445 strain liquid, and Lactobacillus delbrueckii ATCC 9649 strain liquid provided by Example 2-2 respectively. Five inoculated broths were prepared by individually inoculating 4 ml of fermentation broth A with the five mixtures, and the inoculated broths were kept in an anaerobic incubator at 37° C. to conduct fermentation. During the fermentation, fermentation broth samples were taken from each of the broths at 0th, 26th, 42th and 71th hours, respectively. The samples were analyzed by using Agilent 1260 HPLC analysis in combination with Aminex HPX-87H (300×7.8 mm) column so as to calculate the concentrations of glucose, lactic acid, acetic acid, and butyric acid in the fermentation broth, and the yield of butyric acid. The results are shown in Tables 2A and 2B.

TABLE 2A

| | Strain of lactic acid bacterium | | | | | | | | |
|---|---|---|---|---|---|---|---|---|---|
| | Lactobacillus | | | Lactobacillus | | | Lactococcus | | |
| | brevis | | | casei | | | lactis | | |
| | ATCC 14869 | | | BCRC10697 | | | ATCC 19435 | | |
| | Sampling time point (hours) | | | | | | | | |
| | 0 | 26 | 42 | 0 | 26 | 42 | 0 | 26 | 42 |
| Glucose (g/L) | 22.2 | 0 | 0 | 21.9 | 0 | 0 | 21.5 | 2.5 | 0.1 |
| Lactic acid (g/L) | 2.0 | 0 | 0 | 1.3 | 0.3 | 0 | 1.7 | 0 | 0 |
| Acetic acid (g/L) | 7.1 | 6.1 | 6.1 | 14.9 | 9.7 | 9.6 | 14.9 | 11.4 | 10.6 |
| Butyric acid (g/L) | 0.2 | 9.7 | 9.7 | 0.0 | 13.0 | 13.3 | 0.0 | 10.3 | 11.7 |
| Yield of butyric acid (g/g) | | | 0.39 | | | 0.57 | | | 0.5 |

TABLE 2B

| | Strain of lactic acid bacterium | | | | |
|---|---|---|---|---|---|
| | Lactobacillus | | | Lactobacillus | |
| | rhamnosus | | | delbrueckii | |
| | NRRL B-445 | | | ATCC 9649 | |
| | Sampling time point (hours) | | | | |
| | 0 | 26 | 42 | 0 | 71 |
| Glucose (g/L) | 21.7 | 0.0 | 0 | 21.2 | 0 |
| Lactic acid (g/L) | 1.7 | 0.1 | 0 | 1.2 | 0 |
| Acetic acid (g/L) | 15.0 | 10.3 | 10.1 | 14.6 | 10.4 |
| Butyric acid (g/L) | 0.0 | 13.0 | 13.1 | 0 | 13.0 |
| Yield of butyric acid (g/g) | | | 0.56 | | 0.58 |

As shown in Tables 2 A and 2B, in comparison with using a single butyric acid bacterium to conduct the fermentation of glucose-containing substrate (as shown in Table 1A, providing a yield of butyric acid of 0.41), the use of a butyric acid bacterium in combination with a heterofermentative lactic acid bacterium (e.g., Lactobacillus brevis ATCC 14869) could not increase (or could even decrease) the yield of butyric acid but the use of a butyric acid bacterium in combination with a homofermentative lactic acid bacterium (e.g., Lactococcus lactis ATCC 19435 and Lactobacillus delbrueckii ATCC 9649) or a facultative heterofermentative lactic acid bacterium (e.g., Lactobacillus casei ATCC 393, and Lactobacillus rhamnosus NRRL B-445) could effectively increase the yield of butyric acid.

### Example 3: Fermentation of a Glucose-Containing Substrate by Using Clostridium Tyrobutyricum DSM 27751 in Combination with a Facultative Heterofermentative Lactic Acid Bacterium

### 3-1. Selection of Strain

Clostridium tyrobutyricum DSM 27751 was used as the first strain. One of Lactobacillus casei ATCC 393 and Lactobacillus rhamnosus NRRL B-445 was used as the second strain.

### 3-2. Pre-Culture

(a) Clostridium tyrobutyricum DSM 27751: a single colony of this strain was selected and inoculated into 20 ml RCM medium, and the inoculated medium was statically incubated in an anaerobic incubator at 37° C. for about 18 hours.
(b) Lactobacillus casei ATCC 393, and Lactobacillus rhamnosus NRRL B-445: a single colony of each strain was selected and inoculated into 20 ml MRS medium, and the inoculated medium was statically incubated in an anaerobic incubator at 37° C. for about 18 hours.

### 3-3. Fermentation Experiments

Two mixtures were prepared by mixing 10 ml Clostridium tyrobutyricum DSM 27751 strain liquid provided by Example 3-2 with 10 ml each of Lactobacillus casei ATCC 393 strain liquid and Lactobacillus rhamnosus NRRL B-445 strain liquid provided by Example 3-2 respectively. Two inoculated broths were prepared in two air-tight containers by individually inoculating 80 ml of fermentation broth B with the two mixtures. 30 g/L calcium carbonate was added into each of the air-tight containers. Thereafter, the air-tight containers were kept in an anaerobic incubator at 37° C. to conduct fermentation. During the fermentation, fermentation broth samples were taken from each of the broths at 0th, 23th and 55th hours, respectively. The samples were analyzed by using Agilent 1260 HPLC analysis in combination with Aminex HPX-87H (300×7.8 mm) column so as to calculate the concentrations of glucose, lactic acid, acetic acid, propionic acid and butyric acid in the fermentation broth, and the yield of butyric acid. The results are shown in Table 3.

TABLE 3

| | Strain of lactic acid bacterium | | | | | |
|---|---|---|---|---|---|---|
| | Lactobacillus | | | Lactobacillus | | |
| | casei | | | rhamnosus | | |
| | BCRC10697 | | | NRRL B-445 | | |
| | Sampling time point (hours) | | | | | |
| | 0 | 23 | 55 | 0 | 23 | 55 |
| Glucose (g/L) | 55.1 | 0 | 0 | 54.7 | 0.0 | 0.0 |
| Lactic acid (g/L) | 2.7 | 19.9 | 0 | 2.9 | 19.4 | 0.0 |
| Acetic acid (g/L) | 15.3 | 10.2 | 4.5 | 15.3 | 9.2 | 3.9 |
| Propionic acid (g/L) | 0.6 | 0.6 | 0.9 | 0.6 | 0.6 | 0.7 |
| Butyric acid (g/L) | 0.0 | 20.1 | 35.6 | 0.1 | 19.9 | 34.3 |
| Yield of butyric acid (g/g) | | | 0.62 | | | 0.59 |

As shown in Table 3, in comparison with using a single butyric acid bacterium to conduct the fermentation of glucose-containing substrate (as shown in Table 1A, providing a yield of butyric acid of 0.41), the use of a butyric acid bacterium in combination with a facultative heterofermentative lactic acid bacterium (e.g., Lactobacillus casei ATCC 393, and Lactobacillus rhamnosus NRRL B-445) could effectively increase the yield of butyric acid.

### Example 4: Fermentation of a Glucose-Containing Substrate by Using Clostridium Tyrobutyricum DSM 27751 in Combination with a Heterofermentative Lactic Acid Bacterium or a Homofermentative Lactic Acid Bacterium

### 4-1. Selection of Strain

Clostridium tyrobutyricum DSM 27751 was used as the first strain. One of Lactobacillus brevis ATCC 14869 and Bacillus coagulans ATCC 7050 was used as the second strain.

### 4-2. Pre-Culture

(a) Clostridium tyrobutyricum DSM 27751: a single colony of this strain was selected and inoculated into 20 ml RCM medium, and the inoculated medium was statically incubated in an anaerobic incubator at 37° C. for about 18 hours.
(b) Lactobacillus brevis ATCC 14869, and Bacillus coagulans ATCC 7050: a single colony of each strain was selected and inoculated into 5 ml MRS medium, and the inoculated medium was statically incubated in an anaerobic incubator at 37° C. for about 28 hours.

### 4-3. Fermentation Experiments

Three strain liquids, i.e., 0.5 ml of the Clostridium tyrobutyricum DSM 27751 strain liquid provided by Example 4-2 (hereinafter referred to as the “control group”), a mixture of 0.5 ml of Clostridium tyrobutyricum DSM 27751 strain liquid provided by Example 4-2 and 0.5 ml of one of the lactic acid bacteria strain liquid provided by Example 4-2, and a mixture of 0.5 ml of Clostridium tyrobutyricum DSM 27751 strain liquid provided by Example 4-2 and 0.5 ml of the other lactic acid bacteria strain liquid provided by Example 4-2 were prepared. Three inoculated broths were prepared by individually inoculating 4 ml of fermentation broth C with the three strain liquid, and the inoculated broths were kept in an anaerobic incubator at 37° C. to conduct fermentation. During the fermentation, fermentation broth samples were taken from each of the broths at 0th, 25th and 42th hour, respectively. The samples were analyzed by using Agilent 1260 HPLC analysis in combination with Aminex HPX-87H (300×7.8 mm) column so as to calculate the concentrations of glucose, lactic acid, acetic acid, propionic acid and butyric acid in the fermentation broth, and the yield of butyric acid. The results are shown in Table 4.

TABLE 4

| | Strain of lactic acid bacterium | | | | | | | | |
|---|---|---|---|---|---|---|---|---|---|
| | None | | | Lactobacillus brevis | | | Bacillus coagulans | | |
| | (control group) | | | ATCC 14869 | | | ATCC 7050 | | |
| Sampling time point (hours) | 0 | 25 | 42 | 0 | 25 | 42 | 0 | 25 | 42 |
| Glucose (g/L) | 21.2 | 0 | 0 | 22.2 | 0.0 | 0.0 | 22.3 | 0 | 0 |
| Lactic acid (g/L) | 1.6 | 0 | 0 | 2.0 | 0 | 0.0 | 1.6 | 0 | 0 |
| Acetic acid (g/L) | 6.6 | 6.0 | 6.1 | 7.1 | 6.2 | 6.1 | 6.8 | 3.7 | 3.7 |
| Propionic acid (g/L) | 0.7 | 0.5 | 0.6 | 0.7 | 0.5 | 0.5 | 0.6 | 0.5 | 0.5 |
| Butyric acid (g/L) | 0.2 | 9.0 | 8.9 | 0.2 | 9.7 | 9.7 | 0.2 | 11.7 | 11.7 |
| Yield of butyric acid (g/g) | | | 0.39 | | | 0.39 | | | 0.48 |

As shown in Table 4, in comparison with using a single butyric acid bacterium to conduct the fermentation of glucose-containing substrate (i.e., the control group, providing a yield of butyric acid of 0.39), the use of a butyric acid bacterium in combination with a heterofermentative lactic acid bacterium (e.g., Lactobacillus brevis ATCC 14869) could not increase the yield of butyric acid but the use of a butyric acid bacterium in combination with a homofermentative lactic acid bacterium (e.g., Bacillus coagulans ATCC 7050) could effectively increase the yield of butyric acid.

### Example 5: Fermentation of a Glucose-Containing Substrate by Using a Single Clostridium Tyrobutyricum DSM 27751 or a Homofermentative Lactic Acid Bacterium

### 5-1. Selection of Strain

Clostridium tyrobutyricum DSM 27751 was used as the first strain Sporolactobacillus inulinus ATCC 15538 was used as the second strain.

### 5-2. Pre-Culture

(a) Clostridium tyrobutyricum DSM 27751: a single colony of this strain was selected and inoculated into 10 ml RCM medium, and the inoculated medium was statically incubated in an anaerobic incubator at 37° C. for about 18 hours.
(b) Sporolactobacillus inulinus ATCC 15538: a single colony of this strain was selected and inoculated into 10 ml MRS medium, and the inoculated medium was incubated in a shaking incubator (anaerobic) at 200 rpm and 37° C. for about 24 hours.

### 5-3. Fermentation Experiments

Two strain liquid, i.e., 10 ml of the Clostridium tyrobutyricum DSM 27751 strain liquid provided by Example 5-2 and 10 ml of the Sporolactobacillus inulinus ATCC 15538 strain liquid provided by Example 5-2 were prepared. Two inoculated broths were prepared in two air-tight containers by individually inoculating 80 ml of fermentation broth B with the two strain liquid. 10 g/L calcium carbonate was added into each of the air-tight containers. Thereafter, the air-tight containers were kept in an anaerobic incubator at 37° C. to conduct fermentation. During the fermentation, fermentation broth samples were taken from each of the broths at 0th, 24th, 48th and 82th hours, respectively. The samples were analyzed by using Agilent 1260 HPLC analysis in combination with Aminex HPX-87H (300×7.8 mm) column so as to calculate the concentrations of glucose, lactic acid, acetic acid, propionic acid and butyric acid in the fermentation broth, and the pH value and yields of butyric acid and/or lactic acid. The results are shown in Table 5.

TABLE 5

| | Strain | | | | | |
|---|---|---|---|---|---|---|
| | Clostridium tyrobutyricum | | | Sporolactobacillus inulinus | | |
| | DSM 27751 | | | ATCC 15538 | | |
| Sampling time point (hours) | 0 | 24 | 48 | 0 | 24 | 82 |
| Glucose (g/L) | 50.64 | 9.43 | 0 | 52.4 | 33.24 | 12.61 |
| Lactic acid (g/L) | 1.60 | 0 | 0 | 2.64 | 21.57 | 40.47 |
| Acetic acid (g/L) | 13.97 | 13.23 | 11.29 | 14.07 | 13.99 | 14.94 |
| Propionic acid (g/L) | 0.64 | 0.62 | 0.67 | 0.65 | 0.79 | 1.5 |
| Butyric acid (g/L) | 0.44 | 19.55 | 25.75 | 0.00 | 0 | 0 |
| pH value | 7 | | 5.12 | | | 4.13 |
| Yield of butyric acid (g/g) | | | 0.48 | | | |
| Yield of lactic acid (g/g) | | | | | | 0.95 |

As shown in Table 5, the use of a single butyric acid bacterium (e.g., Clostridium tyrobutyricum DSM 27751) in the fermentation of a glucose-containing substrate provided only about 0.48 of the butyric acid yield. On the other hand, if the fermentation of a glucose-containing substrate was conducted by using a single homofermentative lactic acid bacterium (e.g., Sporolactobacillus inulinus ATCC 15538), the yield of lactic acid was about 0.95.

### Example 6: Fermentation of a Glucose-Containing Substrate by Using Clostridium tyrobutyricum DSM 27751 in Combination with a Homofermentative Lactic Acid Bacterium

A mixture was prepared by mixing 10 ml Clostridium tyrobutyricum DSM 27751 strain liquid provided by Example 5-2 with 10 ml Sporolactobacillus inulinus ATCC 15538 strain liquid provided by Example 5-2. An inoculated broth was prepared in an air-tight container by inoculating 80 ml of fermentation broth B with the mixture. 10 g/L calcium carbonate was added into the air-tight container. Thereafter, the air-tight container was kept in an anaerobic incubator at 37° C. to conduct fermentation. During the fermentation, fermentation broth samples were taken from the broth at 0th, 24th and 82th hours, respectively. The samples were analyzed by using Agilent 1260 HPLC analysis in combination with Aminex HPX-87H (300×7.8 mm) column so as to calculate the concentrations of glucose, lactic acid, acetic acid, propionic acid and butyric acid in the fermentation broth, and the pH value and yield of butyric acid. The results are shown in Table 6.

TABLE 6

| | Strain of lactic acid bacterium | | |
|---|---|---|---|
| | Sporolactobacillus inulinus | | |
| | ATCC 15538 | | |
| | Sampling time point (hours) | | |
| | 0 | 24 | 82 |
| Glucose (g/L) | 52.81 | 15.16 | 0.45 |
| Lactic acid (g/L) | 2.61 | 0 | 0 |
| Acetic acid (g/L) | 14.46 | 9.69 | 8.04 |
| Propionic acid (g/L) | 0.64 | 0.65 | 0.65 |
| Butyric acid (g/L) | 0.42 | 23.86 | 32.29 |
| pH value | 7 | | 5.04 |
| Yield of butyric acid (g/g) | | | 0.58 |

As shown in Table 6, in comparison with using a single butyric acid bacterium to conduct the fermentation of glucose-containing substrate (as shown in Table 5, providing a yield of butyric acid of 0.48), the use of a butyric acid bacterium in combination with a homofermentative lactic acid bacterium (e.g., Sporolactobacillus inulinus ATCC 15538) could effectively increase the yield of butyric acid.

### Example 7: Fermentation of a Fructose-Containing Substrate by Using a Single Butyric Acid Bacterium or a Lactic Acid Bacterium

### 7-1. Selection of Strain

To conducted a fermentation by using a single strain, one of Clostridium tyrobutyricum DSM 27751, Lactobacillus casei ATCC 393 and Lactobacillus rhamnosus NRRL B-445 was used.

### 7-2. Pre-Culture

(a) Clostridium tyrobutyricum DSM 27751: a single colony of this strain was selected and inoculated into 20 ml RCM medium, and the inoculated medium was statically incubated in an anaerobic incubator at 37° C. for about 18 hours.
(b) Lactobacillus casei ATCC 393 and Lactobacillus rhamnosus NRRL B-445: a single colony of each strain was selected and inoculated into 20 ml MRS medium, and the inoculated medium was statically incubated in an anaerobic incubator at 37° C. for about 24 hours.

### 7-3. Fermentation Experiments

The strain liquid of Clostridium tyrobutyricum DSM 27751, Lactobacillus casei ATCC 393, and Lactobacillus rhamnosus NRRL B-445 provided by Example 7-2 was respectively inoculated into fermentation broth D at a 10% inoculation rate to provide three fermentation broths each having a final volume of 50 ml. Each of the three inoculated fermentation broths was placed in an air-tight container, and 30 g/L calcium carbonate was then respectively added into each of the air-tight container. Thereafter, the air-tight containers were kept in an anaerobic incubator at 37° C. to conduct fermentation. During the fermentation, fermentation broth samples were taken from each of the broths at 0th, 24th and 48th hours, respectively. The samples were analyzed by using Agilent 1260 HPLC analysis in combination with Aminex HPX-87H (300×7.8 mm) column so as to calculate the concentrations of fructose, lactic acid, acetic acid, propionic acid and butyric acid in the fermentation broth, and the pH value and yields of butyric acid and/or lactic acid. The results are shown in Table 7.

TABLE 7

| | Strain | | | | | | | | |
|---|---|---|---|---|---|---|---|---|---|
| | Clostridium | | | Lactobacillus | | | Lactobacillus | | |
| | tyrobutyricum | | | casei | | | rhamnosus | | |
| | DSM 27751 | | | ATCC 393 | | | NRRL B-445 | | |
| Sampling time point (hours) | 0 | 24 | 48 | 0 | 24 | 48 | 0 | 24 | 48 |
| Fructose (g/L) | 49.1 | 0.8 | 0.4 | 49.1 | 10.9 | 0.4 | 49.2 | 5.8 | 0.5 |
| Lactic acid (g/L) | 1.6 | 0 | 0 | 2.9 | 38.3 | 48.4 | 3.2 | 43.3 | 48.9 |
| Acetic acid (g/L) | 13.8 | 18.1 | 18.2 | 14.0 | 13.7 | 13.8 | 14.0 | 13.9 | 14.4 |
| Propionic acid (g/L) | 0.6 | 0.7 | 0.7 | 0.6 | 0.6 | 0.6 | 0.6 | 0.7 | 0.8 |
| Butyric acid (g/L) | 0.2 | 17.7 | 17.9 | 0.00 | 0.2 | 0.1 | 0.0 | 0.2 | 0.1 |
| pH value | 6.2 | | 5.2 | 5.9 | | 5.1 | 5.9 | | 5.2 |
| Yield of butyric acid (g/g) | | | 0.35 | | | | | | |
| Yield of lactic acid (g/g) | | | | | | 0.93 | | | 0.94 |

As shown in Table 7, the use of a single butyric acid bacterium (e.g., Clostridium tyrobutyricum DSM 27751) in the fermentation of a fructose-containing substrate provided only about 0.35 of the butyric acid yield. On the other hand, if the fermentation of a fructose-containing substrate was conducted by using a single lactic acid bacterium (e.g., Lactobacillus casei ATCC 393, and Lactobacillus rhamnosus NRRL B-445), the yield of lactic acid was at least 0.9.

### Example 8: Fermentation of a Fructose-Containing Substrate by Using Clostridium Tyrobutyricum DSM 27751 in Combination with a Facultative Heterofermentative Lactic Acid Bacterium

Strain liquid of Clostridium tyrobutyricum DSM 27751 provided by Example 7-2 was inoculated into fermentation broth D at a 10% inoculation rate, and then, the strain liquid of Lactobacillus casei ATCC 393 or Lactobacillus rhamnosus NRRL B-445 provided by Example 7-2 was inoculated thereinto at a 10% inoculation rate to provide a fermentation broth having a final volume of 50 ml. The fermentation broth was placed in an air-tight container, and 30 g/L calcium carbonate was then added into the air-tight containers. Thereafter, the air-tight containers was kept in an anaerobic incubator at 37° C. to conduct fermentation. During the fermentation, fermentation broth samples were taken from the broth at 0th, 48th and 54th hours, respectively. The samples were analyzed by using Agilent 1260 HPLC analysis in combination with Aminex HPX-87H (300×7.8 mm) column so as to calculate the concentrations of glucose, fructose, lactic acid, acetic acid, propionic acid and butyric acid in the fermentation broth, and the pH value and yield of butyric acid. The results are shown in Table 8.

TABLE 8

| | Strain of lactic acid bacterium | | | | | |
|---|---|---|---|---|---|---|
| | Lactobacillus casei | | | Lactobacillus rhamnosus | | |
| | ATCC 393 | | | NRRL B-445 | | |
| Sampling time point (hours) | 0 | 48 | 54 | 0 | 48 | 54 |
| Glucose (g/L) | 0.1 | 0 | 0 | 0 | 0 | 0 |
| Fructose (g/L) | 49.7 | 0.4 | 0.4 | 49.6 | 0.8 | 0.6 |
| Lactic acid (g/L) | 2.9 | 4.5 | 0 | 3.2 | 20.4 | 0 |
| Acetic acid (g/L) | 14.2 | 7.6 | 6.6 | 14.2 | 9.7 | 4.4 |
| Propionic acid (g/L) | 0.6 | 0.6 | 0.9 | 0.6 | 0.7 | 0.8 |
| Butyric acid (g/L) | 0.2 | 27.3 | 30.6 | 0.2 | 18.0 | 32.3 |
| pH value | 5.8 | | 6.5 | 5.8 | | 6.3 |
| Yield of butyric acid (g/g) | | | 0.66 | | | 0.62 |

As shown in Table 8, in comparison with using a single butyric acid bacterium to conduct the fermentation of fructose-containing substrate (as shown in Table 7, providing a yield of butyric acid of 0.35), the use of a butyric acid bacterium in combination with a facultative heterofermentative lactic acid bacterium (e.g., Lactobacillus casei ATCC 393, and Lactobacillus rhamnosus NRRL B-445) could effectively increase the yield of butyric acid.

### Example 9: Fermentation of a Xylose-Containing Substrate by Using a Single Butyric Acid Bacterium or a Lactic Acid Bacterium

Strain liquids of Clostridium tyrobutyricum DSM 27751, Lactobacillus casei ATCC 393, and Lactobacillus rhamnosus NRRL B-445 provided by Example 7-2 were individually inoculated into 5 ml fermentation broth E at a 10% inoculation rate, and then, the three inoculated fermentation broths were kept in an anaerobic incubator at 37° C. to conduct fermentation. During the fermentation, fermentation broth samples were taken from each of the broths at 0th, 23th and 53th hours, respectively. The samples were analyzed by using Agilent 1260 HPLC analysis in combination with Aminex HPX-87H (300×7.8 mm) column so as to calculate the concentrations of glucose, xylose, lactic acid, acetic acid, propionic acid and butyric acid in the fermentation broth. The results are shown in Table 9.

TABLE 9

| | Strain | | | | | | | | |
|---|---|---|---|---|---|---|---|---|---|
| | Clostridium | | | Lactobacillus | | | Lactobacillus | | |
| | tyrobutyricum | | | casei | | | rhamnosus | | |
| | DSM 27751 | | | ATCC 393 | | | NRRL B-445 | | |
| Sampling time point (hours) | 0 | 23 | 53 | 0 | 23 | 53 | 0 | 23 | 53 |
| Glucose (g/L) | 0 | 0 | 0 | 0.5 | 0 | 0 | 0 | 0 | 0 |
| Xylose (g/L) | 19.8 | 19.4 | 19.5 | 19.9 | 19.5 | 19.5 | 19.8 | 19.4 | 19.5 |
| Lactic acid (g/L) | 1.5 | 0 | 0 | 2.5 | 3.4 | 3.5 | 2.9 | 3.5 | 3.4 |
| Acetic acid (g/L) | 6.4 | 5.9 | 6.0 | 6.5 | 6.7 | 6.8 | 6.5 | 6.6 | 6.7 |
| Propionic acid (g/L) | 0.6 | 0.6 | 0.5 | 0.5 | 0.6 | 0.6 | 0.6 | 0.6 | 0.6 |
| Butyric acid (g/L) | 0.2 | 1.5 | 1.5 | 0 | 0 | 0 | 0 | 0 | 0 |

As shown in Table 9, Clostridium tyrobutyricum DSM 27751 cannot ferment xylose into butyric acid, and Lactobacillus casei ATCC 393 and Lactobacillus rhamnosus NRRL B-445 cannot ferment xylose into lactic acid.

### Example 10: Fermentation of a Xylose-Containing Substrate by Using Clostridium Tyrobutyricum DSM 27751 in Combination with a Facultative Heterofermentative Lactic Acid Bacterium

### 10-1. Selection of Strain

Clostridium tyrobutyricum DSM 27751 was used as the first strain. One of Lactobacillus casei ATCC 393 and Lactobacillus rhamnosus NRRL B-445 was used as the second strain. 10-2. Pre-culture
(a) Clostridium tyrobutyricum DSM 27751: a single colony of this strain was selected and inoculated into 5 ml RCM medium, and the inoculated medium was statically incubated in an anaerobic incubator at 37° C. for about 18 hours.
(b) Lactobacillus casei ATCC 393 and Lactobacillus rhamnosus NRRL B-445: a single colony of each strain was selected and inoculated into 5 ml MRS medium, and the inoculated medium was statically incubated in an anaerobic incubator at 37° C. for about 25 hours.

### 10-3. Fermentation Experiments

Two mixtures were prepared by mixing 0.5 ml Clostridium tyrobutyricum DSM 27751 strain liquid provided by Example 10-2 with 0.5 ml each of Lactobacillus casei ATCC 393 strain liquid and Lactobacillus rhamnosus NRRL B-445 strain liquid provided by Example 10-2 respectively. Two inoculated broths were prepared by individually inoculating into 4 ml of fermentation broth E with the two mixtures, and the inoculated broths were kept in an anaerobic incubator at 37° C. to conduct fermentation. During the fermentation, fermentation broth samples were taken from each of the broths at 0th, 23th and 53th hours, respectively. The samples were analyzed by using Agilent 1260 HPLC analysis in combination with Aminex HPX-87H (300×7.8 mm) column so as to calculate the concentrations of xylose, lactic acid, acetic acid, propionic acid and butyric acid in the fermentation broth. The results are shown in Table 10.

TABLE 10

| | Strain of lactic acid bacterium | | | | | |
|---|---|---|---|---|---|---|
| | Lactobacillus casei | | | Lactobacillus rhamnosus | | |
| | ATCC 393 | | | NRRL B-445 | | |
| Sampling time point (hours) | 0 | 23 | 53 | 0 | 23 | 53 |
| Xylose (g/L) | 19.7 | 19.4 | 19.4 | 19.7 | 19.4 | 19.4 |
| Lactic acid (g/L) | 2.5 | 0 | 0 | 3.0 | 0 | 0 |
| Acetic acid (g/L) | 6.8 | 5.8 | 6.0 | 6.8 | 5.9 | 6.0 |
| Propionic acid (g/L) | 0.6 | 0.6 | 0.6 | 0.6 | 0.6 | 0.6 |
| Butyric acid (g/L) | 0.2 | 2.8 | 2.9 | 0.2 | 2.7 | 2.8 |

As shown in Table 10, the use of a butyric acid bacterium in combination with a facultative heterofermentative lactic acid bacterium cannot ferment xylose into butyric acid or lactic acid.

### Example 11: Fermentation of a Sucrose-Containing Substrate by Using a Single Butyric Acid Bacterium or a Lactic Acid Bacterium

Three inoculated broths were prepared by individually inoculating 5 ml of fermentation broth F with strain liquids of Clostridium tyrobutyricum DSM 27751, Lactobacillus casei ATCC 393, and Lactobacillus rhamnosus NRRL B-445 provided by Example 7-2 at a 10% inoculation rate. The three inoculated fermentation broths were kept in an anaerobic incubator at 37° C. to conduct fermentation. During the fermentation, fermentation broth samples were taken from each of the broths at 0th, 24th and 48th hours, respectively. The samples were analyzed by using Agilent 1260 HPLC analysis in combination with Aminex HPX-87H (300×7.8 mm) column so as to calculate the concentrations of sucrose (based on the concentrations of glucose and fructose), lactic acid, acetic acid, propionic acid and butyric acid in the fermentation broth, and the yields of butyric acid and/or lactic acid. The results are shown in Table 11.

TABLE 11

| | Strain | | | | | | | | |
|---|---|---|---|---|---|---|---|---|---|
| | Clostridium | | | Lactobacillus | | | Lactobacillus | | |
| | tyrobutyricum | | | casei | | | rhamnosus | | |
| | DSM 27751 | | | ATCC 393 | | | NRRL B-445 | | |
| Sampling time point (hours) | 0 | 24 | 48 | 0 | 24 | 48 | 0 | 24 | 48 |
| Glucose (g/L) | 10.4 | 6.0 | 6.0 | 10.4 | 5.7 | 5.6 | 10.4 | 5.9 | 5.8 |
| Fructose (g/L) | 10.1 | 4.8 | 4.8 | 10.1 | 4.6 | 4.6 | 10.1 | 4.8 | 4.7 |
| Lactic acid (g/L) | 1.5 | 0 | 0 | 2.8 | 12.7 | 13.3 | 3.0 | 12.5 | 12.6 |
| Acetic acid (g/L) | 6.4 | 6.8 | 6.8 | 6.5 | 6.5 | 6.5 | 6.5 | 6.6 | 6.5 |
| Propionic acid (g/L) | 0.5 | 0.6 | 0.6 | 0.5 | 0.6 | 0.6 | 0.6 | 0.6 | 0.6 |
| Butyric acid (g/L) | 0.2 | 4.8 | 4.7 | 0.0 | 0.0 | 0.0 | 0.0 | 0.1 | 0.1 |
| Yield of butyric acid (g/g) | | | 0.40 | | | | | | |
| Yield of lactic acid (g/g) | | | | | | 1.01 | | | 0.96 |

As shown in Table 11, the use of a single butyric acid bacterium (e.g., Clostridium tyrobutyricum DSM 27751) in the fermentation of a sucrose-containing substrate provided only about 0.4 of the butyric acid yield. On the other hand, if the fermentation of a sucrose-containing substrate was conducted by using a single lactic acid bacterium (e.g., Lactobacillus casei ATCC 393 and Lactobacillus rhamnosus NRRL B-445), the yield of lactic acid was nearly 1.

### Example 12: Fermentation of a Sucrose-Containing Substrate by Using Clostridium Tyrobutyricum DSM 27751 in Combination with a Facultative Heterofermentative Lactic Acid Bacterium

### 12-1. Selection of Strain

Clostridium tyrobutyricum DSM 27751 was used as the first strain. One of Lactobacillus casei ATCC 393 and Lactobacillus rhamnosus NRRL B-445 was used as the second strain. 12-2. Pre-culture
(a) Clostridium tyrobutyricum DSM 27751: a single colony of this strain was selected and inoculated into 5 ml RCM medium, and the inoculated medium was statically incubated in an anaerobic incubator at 37° C. for about 18 hours.
(b) Lactobacillus casei ATCC 393 and Lactobacillus rhamnosus NRRL B-445: a single colony of each strain was selected and inoculated into 5 ml MRS medium, and the inoculated medium was statically incubated in an anaerobic incubator at 37° C. for about 24 hours.

### 12-3. Fermentation Experiments

Two mixtures were prepared by mixing 0.5 ml Clostridium tyrobutyricum DSM 27751 strain liquid provided by Example 12-2 with 0.5 ml each of Lactobacillus casei ATCC 393 strain liquid and Lactobacillus rhamnosus NRRL B-445 strain liquid provided by Example 12-2 respectively. Two inoculated broths were prepared by individually inoculating 4 ml of fermentation broth F with the two mixtures, and the broths were kept in an anaerobic incubator at 37° C. to conduct fermentation. During the fermentation, fermentation broth samples were taken from each of the broths at 0th, 48th and 120th hours, respectively. The samples were analyzed by using Agilent 1260 HPLC analysis in combination with Aminex HPX-87H (300×7.8 mm) column so as to calculate the concentrations of sucrose (based on the concentrations of glucose and fructose), lactic acid, acetic acid, propionic acid and butyric acid in the fermentation broth, and the yield of butyric acid. The results are shown in Table 12.

TABLE 12

| | Strain of lactic acid bacterium | | | | | |
|---|---|---|---|---|---|---|
| | Lactobacillus casei | | | Lactobacillus rhamnosus | | |
| | ATCC 393 | | | NRRL B-445 | | |
| Sampling time point (hours) | 0 | 48 | 120 | 0 | 48 | 120 |
| Glucose (g/L) | 10.4 | 5.6 | 5.0 | 10.4 | 5.8 | 2.9 |
| Fructose (g/L) | 10.1 | 4.6 | 4.8 | 10.1 | 4.7 | 2.7 |
| Lactic acid (g/L) | 2.8 | 0 | 0.0 | 3.0 | 0 | 0.7 |
| Acetic acid (g/L) | 6.7 | 4.3 | 4.2 | 6.8 | 3.9 | 3.1 |
| Propionic acid (g/L) | 0.5 | 0.6 | 0.6 | 0.5 | 0.6 | 0.6 |
| Butyric acid (g/L) | 0.1 | 8.3 | 8.5 | 0.1 | 8.4 | 10.3 |
| Yield of butyric acid (g/g) | | | 0.62 | | | 0.59 |

As shown in Table 12, in comparison with using a single butyric acid bacterium to conduct the fermentation of sucrose-containing substrate (as shown in Table 11, providing a yield of butyric acid of 0.4), the use of a butyric acid bacterium in combination with a facultative heterofermentative lactic acid bacterium (e.g., Lactobacillus casei ATCC 393, and Lactobacillus rhamnosus NRRL B-445) could effectively increase the yield of butyric acid.

### Example 13: Fermentation of a Molasses-Containing Substrate by Using a Single Butyric Acid Bacterium or a Lactic Acid Bacterium

Three inoculated broths were prepared by individually inoculating 5 ml of fermentation broth G with strain liquids of Clostridium tyrobutyricum DSM 27751, Lactobacillus casei ATCC 393 and Lactobacillus rhamnosus NRRL B-445 provided by Example 7-2 at a 10% inoculation rate. The three inoculated fermentation broths were kept in an anaerobic incubator at 37° C. to conduct fermentation. During the fermentation, fermentation broth samples were taken from each of the broths at 0th, 24th and 72th hours, respectively. The samples were analyzed by using Agilent 1260 HPLC analysis in combination with Aminex HPX-87H (300×7.8 mm) column so as to calculate the concentrations of molasses (based on the concentrations of glucose and fructose), lactic acid, acetic acid, propionic acid and butyric acid in the fermentation broth, and the yields of butyric acid and/or lactic acid. The results are shown in Table 13.

TABLE 13

| | Strain | | | | | | | | |
|---|---|---|---|---|---|---|---|---|---|
| | Clostridium | | | Lactobacillus | | | Lactobacillus | | |
| | tyrobutyricum | | | casei | | | rhamnosus | | |
| | DSM 27751 | | | ATCC 393 | | | NRRL B-445 | | |
| Sampling time point (hours) | 0 | 24 | 72 | 0 | 24 | 72 | 0 | 24 | 72 |
| Glucose (g/L) | 5.2 | 3.7 | 3.7 | 5.4 | 3.8 | 3.5 | 5.1 | 3.8 | 3.2 |
| Fructose (g/L) | 6.0 | 3.8 | 3.8 | 6.0 | 3.7 | 3.3 | 6.0 | 3.7 | 3.0 |
| Lactic acid (g/L) | 1.7 | 0.0 | 0.0 | 3.2 | 7.5 | 7.5 | 3.5 | 7.2 | 8.0 |
| Acetic acid (g/L) | 12.7 | 12.5 | 12.5 | 12.9 | 13.1 | 13.1 | 12.8 | 13.0 | 13.1 |
| Propionic acid (g/L) | 0.6 | 0.6 | 0.6 | 0.6 | 0.6 | 0.6 | 0.6 | 0.6 | 0.6 |
| Butyric acid (g/L) | 1.6 | 2.6 | 2.6 | 0.0 | 0.0 | 0.0 | 0.0 | 0.0 | 0.0 |
| Yield of butyric acid (g/g) | | | 0.20 | | | | | | |
| Yield of lactic acid (g/g) | | | | | | 0.95 | | | 0.94 |

As shown in Table 13, the use of a single butyric acid bacterium (e.g., Clostridium tyrobutyricum DSM 27751) in the fermentation of a molasses-containing substrate provided only about 0.2 of the butyric acid yield. On the other hand, if the fermentation of a molasses-containing substrate was conducted by using a single lactic acid bacterium (e.g., Lactobacillus casei ATCC 393, and Lactobacillus rhamnosus NRRL B-445), the yield of lactic acid was about 0.95.

### Example 14: Fermentation of a Molasses-Containing Substrate by Using Clostridium Tyrobutyricum DSM 27751 in Combination with a Facultative Heterofermentative Lactic Acid Bacterium

### 14-1. Selection of Strain

Clostridium tyrobutyricum DSM 27751 was used as the first strain. One of Lactobacillus casei ATCC 393 and Lactobacillus rhamnosus NRRL B-445 was used as the second strain. 14-2. Pre-culture
(a) Clostridium tyrobutyricum DSM 27751: a single colony of this strain was selected and inoculated into 5 ml RCM medium, and the inoculated medium was statically incubated in an anaerobic incubator at 37° C. for about 18 hours.
(b) Lactobacillus casei ATCC 393 and Lactobacillus rhamnosus NRRL B-445: a single colony of each strain was selected and inoculated into 5 ml MRS medium, and the inoculated medium was statically incubated in an anaerobic incubator at 37° C. for about 24 hours.

### 14-3. Fermentation Experiments

Two mixtures were prepared by mixing 0.5 ml Clostridium tyrobutyricum DSM 27751 strain liquid provided by Example 14-2 with 0.5 ml each of Lactobacillus casei ATCC 393 strain liquid and Lactobacillus rhamnosus NRRL B-445 strain liquid provided by Example 14-2. Two inoculated broths were prepared by individually inoculating 4 ml of fermentation broth H with the two mixtures, and the inoculated broths were kept in an anaerobic incubator at 37° C. to conduct fermentation. During the fermentation, fermentation broth samples were taken from each of the broths at 0th, 24th and 72th hours, respectively. The samples were analyzed by using Agilent 1260 HPLC analysis in combination with Aminex HPX-87H (300×7.8 mm) column so as to calculate the concentrations of molasses (based on the concentrations of glucose and fructose), lactic acid, acetic acid, propionic acid and butyric acid in the fermentation broth, and the yield of butyric acid. The results are shown in Table 14.

TABLE 14

| | Strain of lactic acid bacterium | | | | | |
|---|---|---|---|---|---|---|
| | Lactobacillus casei | | | Lactobacillus rhamnosus | | |
| | ATCC 393 | | | NRRL B-445 | | |
| Sampling time point (hours) | 0 | 24 | 72 | 0 | 24 | 72 |
| Glucose (g/L) | 5.5 | 3.6 | 3.6 | 5.2 | 3.6 | 0.3 |
| Fructose (g/L) | 6.0 | 3.4 | 3.3 | 6.0 | 3.4 | 0.2 |
| Lactic acid (g/L) | 3.2 | 0.0 | 0.0 | 3.4 | 0.0 | 0.0 |
| Acetic acid (g/L) | 13.1 | 11.5 | 11.4 | 13.1 | 11.4 | 10.3 |
| Propionic acid (g/L) | 0.6 | 0.6 | 0.6 | 0.6 | 0.6 | 0.7 |
| Butyric acid (g/L) | 0.2 | 5.0 | 5.1 | 0.2 | 5.0 | 7.8 |
| Yield of butyric acid (g/g) | | | 0.64 | | | 0.54 |

As shown in Table 14, in comparison with using a single butyric acid bacterium to conduct the fermentation of molasses-containing substrate (as shown in Table 13, providing a yield of butyric acid of 0.2), the use of a butyric acid bacterium in combination with a facultative heterofermentative lactic acid bacterium (e.g., Lactobacillus casei ATCC 393, and Lactobacillus rhamnosus NRRL B-445) could effectively increase the yield of butyric acid.

### Example 15: Fermentation of a Glucose-Containing Substrate by Using a Single Clostridium Tyrobutyricum ATCC 25755

### 15-1. Selection of Strain

To conducted a fermentation by using a single strain, Clostridium tyrobutyricum ATCC 25755 was used.

### 15-2. Pre-Culture

Clostridium tyrobutyricum ATCC 25755: a single colony of this strain was selected and inoculated into 28 ml RCM medium, and the inoculated medium was statically incubated in an anaerobic incubator at 37° C. for about 18 hours.

### 15-3. Fermentation Experiments

Strain liquid of Clostridium tyrobutyricum ATCC 25755 provided by Example 15-2 was inoculated into fermentation broth B at a 10% inoculation rate to provide a fermentation broth having a final volume of 50 ml. The fermentation broth was placed in an air-tight container, and 30 g/L calcium carbonate was then added into the air-tight container. Thereafter, the air-tight container was kept in an anaerobic incubator at 37° C. to conduct fermentation. During the fermentation, fermentation broth samples were taken from the broth at 0th, 24th and 48th hours, respectively. The samples were analyzed by using Agilent 1260 HPLC analysis in combination with Aminex HPX-87H (300×7.8 mm) column so as to calculate the concentrations of glucose, lactic acid, acetic acid, propionic acid and butyric acid in the fermentation broth, and the pH value and yield of butyric acid. The results are shown in Table 15.

TABLE 15

| | Strain | | |
|---|---|---|---|
| | Clostridium tyrobutyricum | | |
| | ATCC 25755 | | |
| | Sampling time point (hours) | | |
| | 0 | 24 | 48 |
| Glucose (g/L) | 51.6 | 48.7 | 47.3 |
| Lactic acid (g/L) | 1.5 | 0.4 | 0 |
| Acetic acid (g/L) | 13.5 | 13.6 | 13.5 |
| Propionic acid (g/L) | 0.6 | 0.6 | 0.6 |
| Butyric acid (g/L) | 0.5 | 2.1 | 3.0 |
| pH value | 6.2 | | |
| Yield of butyric acid (g/g) | | | 0.44 |

As shown in Table 15, the use of another butyric acid bacterium (e.g., Clostridium tyrobutyricum ATCC 25755) in the fermentation of a glucose-containing substrate provided about 0.44 of the butyric acid yield.

### Example 16: Fermentation of a Glucose-Containing Substrate by Using Clostridium Tyrobutyricum ATCC 25755 in Combination with a Homofermentative Lactic Acid Bacterium, a Facultative Heterofermentative Lactic Acid Bacterium, or a Heterofermentative Lactic Acid Bacterium

### 16-1. Selection of Strain

Clostridium tyrobutyricum ATCC 25755 was used as the first strain. One of Lactobacillus brevis ATCC 14869, Lactobacillus casei ATCC 393, Bacillus coagulans ATCC 7050 and Lactobacillus rhamnosus NRRL B-445 was used as the second strain. 16-2. Pre-culture
(a) Clostridium tyrobutyricum ATCC 25755: a single colony of this strain was selected and inoculated into 28 ml RCM medium, and the inoculated medium was statically incubated in an anaerobic incubator at 37° C. for about 18 hours.
(b) Lactobacillus brevis ATCC 14869, Lactobacillus casei ATCC 393, Bacillus coagulans ATCC 7050 and Lactobacillus rhamnosus NRRL B-445: a single colony of each strain was selected and inoculated into 20 ml MRS medium, and the inoculated medium was statically incubated in an anaerobic incubator at 37° C. for about 24 hours.

### 16-3. Fermentation Experiments

Strain liquid of Clostridium tyrobutyricum ATCC 25755 provided by Example 16-2 was inoculated into fermentation broth B at a 10% inoculation rate, and then, strain liquid of each of Lactobacillus brevis ATCC 14869, Lactobacillus casei ATCC 393, Bacillus coagulans ATCC 7050 and Lactobacillus rhamnosus NRRL B-445 provided by Example 16-2 was inoculated thereinto at a 10% inoculation rate to provide a fermentation broth having a final volume of 50 ml. The fermentation broth was placed in an air-tight container, and 30 g/L calcium carbonate was then added into the container. Thereafter, the air-tight containers were kept in an anaerobic incubator at 37° C. to conduct fermentation. During the fermentation, fermentation broth samples were taken from each of the broth at 0th, 24th, 48th and 144th hours, respectively. The samples were analyzed by using Agilent 1260 HPLC analysis in combination with Aminex HPX-87H (300×7.8 mm) column so as to calculate the concentrations of glucose, lactic acid, acetic acid, propionic acid and butyric acid in the fermentation broth, and the pH value and yield of butyric acid. The results are shown in Tables 16A and 16B.

TABLE 16A

| | Strain of lactic acid bacterium | | | | | | | |
|---|---|---|---|---|---|---|---|---|
| | Lactobacillus brevis | | | | Lactobacillus casei | | | |
| | ATCC 14869 | | | | ATCC 393 | | | |
| Sampling time point (hours) | 0 | 24 | 48 | 144 | 0 | 24 | 48 | 144 |
| Glucose (g/L) | 53.1 | 45.9 | 40.8 | 31.1 | 51.8 | 0 | 0 | 0 |
| Lactic acid (g/L) | 1.6 | 0.5 | 0.4 | 0 | 3.0 | 48.0 | 42.2 | 0 |
| Acetic acid (g/L) | 14.1 | 13.8 | 13.9 | 13.7 | 14.0 | 13.7 | 12.0 | 1.1 |
| Propionic acid (g/L) | 0.6 | 0.6 | 0 | 0.6 | 0.6 | 0.7 | 0.6 | 0.7 |
| Butyric acid (g/L) | 0.2 | 3.8 | 6 | 10.1 | 0.1 | 2.0 | 6.4 | 37.7 |
| pH value | 6.3 | | | 5.2 | 5.9 | | | |
| Yield of butyric acid (g/g) | | | | 0.42 | | | | 0.68 |

TABLE 16B

| | Strain of lactic acid bacterium | | | | | | | |
|---|---|---|---|---|---|---|---|---|
| | Bacillus coagulans | | | | Lactobacillus rhamnosus | | | |
| | ATCC 7050 | | | | NRRL B-445 | | | |
| Sampling time point (hours) | 0 | 24 | 48 | 144 | 0 | 24 | 48 | 144 |
| Glucose (g/L) | 52.3 | 39.8 | 26.5 | 6.8 | 51.9 | 0 | 0 | 0 |
| Lactic acid (g/L) | 2.4 | 6.4 | 10.7 | 13.2 | 3.1 | 42.7 | 7.0 | 0 |
| Acetic acid (g/L) | 14.0 | 13.0 | 11.6 | 8.8 | 14.0 | 12.9 | 3.4 | 1.4 |
| Propionic acid (g/L) | 0.6 | 0.6 | 0.6 | 0.4 | 0.6 | 0.6 | 0.6 | 0.7 |
| Butyric acid (g/L) | 0.1 | 4.9 | 10.0 | 21.3 | 0.1 | 4.9 | 30.6 | 37.1 |
| pH value | 6.0 | | | 5.1 | 5.9 | | | |
| Yield of butyric acid (g/g) | | | | | | | | 0.67 |

As shown in Tables 16A and 16B, in comparison with using a single butyric acid bacterium to conduct the fermentation of glucose-containing substrate (as shown in Table 15, providing a yield of butyric acid of 0.44), the use of a butyric acid bacterium in combination with a heterofermentative lactic acid bacterium (e.g., Lactobacillus brevis ATCC 14869) could not increase (or could even decrease) the yield of butyric acid but the use of a butyric acid bacterium in combination with a homofermentative lactic acid bacterium (e.g., Bacillus coagulans ATCC 7050) or a facultative heterofermentative lactic acid bacterium (e.g., Lactobacillus casei ATCC 393, and Lactobacillus rhamnosus NRRL B-445) could effectively increase the yield of butyric acid.

### Example 17: Fermentation of a Glucose-Containing Substrate by Adding Clostridium Tyrobutyricum DSM 27751 and Lactobacillus rhamnosus NRRL B-445 at Different Time Points

### 17-1. Selection of Strain

Clostridium tyrobutyricum DSM 27751 was used as the first strain. Lactobacillus rhamnosus NRRL B-445 was used as the second strain.

### 17-2. Pre-Culture

(a) Clostridium tyrobutyricum DSM 27751: a single colony of this strain was selected and inoculated into 10 ml RCM medium, and the inoculated medium was statically incubated in an anaerobic incubator at 37° C. for about 8 hours. Thereafter, the fermentation broth thus obtained was inoculated into 90 ml RCM medium, and the inoculated medium was incubated in a shaking incubator (anaerobic) at 37° C. for about 16 hours.
(b) Lactobacillus rhamnosus NRRL B-445: a single colony of this strain was selected and inoculated into 10 ml MRS medium, and the inoculated medium was statically incubated in an anaerobic incubator at 37° C. for about 16 hours. Thereafter, the fermentation broth thus obtained was inoculated into 90 ml MRS medium, and the inoculated medium was incubated in a shaking incubator (anaerobic) at 37° C. for about 8 hours.

### 17-3. Fermentation Experiments

Three air-tight containers were prepared. Each of the three containers were added with 80 ml fermentation broth I, and then the containers were labeled as “group A,” “group B,” and “group C.” 10 ml Lactobacillus rhamnosus NRRL B-445 strain liquid provided by Example 17-2 and 30 g/L calcium carbonate were added into each of the air-tight containers. The three air-tight containers were all kept in an anaerobic incubator at 37° C., and 10 ml Clostridium tyrobutyricum DSM 27751 strain liquid provided by Example 17-2 was respectively added into the air-tight container of “group A” at 16th hour, added into the air-tight container of “group B” at 20th hour, and added into the air-tight container of “group C” at 24th hour. Samples were taken from the three air-tight containers at 0th, 16th, 20th, 24th, 48th and 112th hour, respectively. The samples were analyzed by using Agilent 1260 HPLC analysis in combination with Aminex HPX-87H (300×7.8 mm) column so as to calculate the concentrations of glucose, lactic acid, acetic acid, propionic acid and butyric acid in the fermentation broth, and the pH value and yield of butyric acid. The results are shown in Tables 17A to 17C.

TABLE 17A

| | Air-tight container | | | | | |
|---|---|---|---|---|---|---|
| | A (Clostridium tyrobutyricum DSM 27751 | | | | | |
| | was added therein at 16 hours) | | | | | |
| Sampling time point (hours) | 0 | 16 | 20 | 24 | 48 | 112 |
| Glucose (g/L) | 58.0 | 19.9 | 10.1 | 1.2 | 0 | 0 |
| Lactic acid (g/L) | 2.0 | 30.2 | 39.4 | 47.6 | 48.0 | 1.9 |
| Acetic acid (g/L) | 13.9 | 12.8 | 12.8 | 12.8 | 12.5 | 0 |
| Propionic acid (g/L) | 0 | 0 | 0 | 0 | 0 | 0 |
| Butyric acid (g/L) | 0 | 0.1 | 0.1 | 0.1 | 0.7 | 32.7 |
| pH value | | | | | | 6.4 |
| Yield of butyric acid (g/g) | | | | | | 0.63 |

TABLE 17B

| | Air-tight container | | | | | |
|---|---|---|---|---|---|---|
| | B (Clostridium tyrobutyricum DSM 27751 | | | | | |
| | was added therein at 20 hours) | | | | | |
| Sampling time point (hours) | 0 | 16 | 20 | 24 | 48 | 112 |
| Glucose (g/L) | 57.7 | 28.8 | 18.5 | 9.4 | 0 | 0 |
| Lactic acid (g/L) | 2.0 | 27.2 | 31.1 | 39.2 | 44.92 | 0.3 |
| Acetic acid (g/L) | 13.9 | 14.0 | 12.8 | 12.7 | 11.9 | 0.2 |
| Propionic acid (g/L) | 0 | 0 | 0 | 0 | 0 | 0 |
| Butyric acid (g/L) | 0 | 0 | 0 | 0 | 2.3 | 34.8 |
| pH value | | | | | | 6.4 |
| Yield of butyric acid (g/g) | | | | | | 0.65 |

TABLE 17C

| | Air-tight container | | | | | |
|---|---|---|---|---|---|---|
| | C (Clostridium tyrobutyricum DSM 27751 | | | | | |
| | was added therein at 24 hours) | | | | | |
| Sampling time point (hours) | 0 | 16 | 20 | 24 | 48 | 112 |
| Glucose (g/L) | 57.8 | 29.8 | 21.8 | 13.1 | 0 | 0 |
| Lactic acid (g/L) | 2.0 | 26.2 | 33.7 | 36.2 | 41.8 | 0 |
| Acetic acid (g/L) | 13.9 | 14.0 | 14.0 | 12.7 | 11.0 | 0.3 |
| Propionic acid (g/L) | 0 | 0 | 0 | 0 | 0 | 0 |
| Butyric acid (g/L) | 0 | 0.0 | 0.0 | 0.0 | 4.7 | 34.6 |
| pH value | | | | | | 6.4 |
| Air-tight container | | | | | | 0.64 |

As shown in Tables 17A to 17C, the use of a butyric acid bacterium in combination with a homofermentative lactic acid bacterium or a facultative heterofermentative lactic acid bacterium (e.g., Lactobacillus rhamnosus NRRL B-445), no matter the two strains were mixed at which time point, the yield of butyric acid was high.

In Tables 1A to 17C, the amount of lactic acid detected from the samples of 0th hour was provided by the inoculated strain liquid.

The results of the above Examples clearly indicate that, as compared to the prior art's method using a single butyric acid bacterium, the method of the present invention using a butyric acid bacterium in combination with a homofermentative lactic acid bacterium or with a facultative heterofermentative lactic acid bacterium in the fermentation of a saccharide-containing substrate could provide a better yield of butyric acid and a decreased production of by-product. Furthermore, there is no particular limitation to the time point for mixing the butyric acid bacterium with a homofermentative lactic acid bacterium or with a facultative heterofermentative lactic acid bacterium.

### DEPOSIT OF BIOLOGICAL MATERIAL

Clostridium tyrobutyricum DSM 27751: DE Germany German Collection of Microorganisms and Cell Cultures (Deutsche Sammlung von Mikroorganismen and Zellkulturen GmbH, DSMZ); Address: InhoffenstraBe 7 B, 38124 Braunschweig, GERMANY; Accession number: DSM 27751.

Clostridium tyrobutyricum ATCC 25755: American Type Culture Collection (ATCC); Address: 10801 University Boulevard Manassas, Va. 20110 USA; Accession number: ATCC 25755.

Lactobacillus casei: American Type Culture Collection (ATCC); Address: 10801 University Boulevard Manassas, Va. 20110 USA; Accession number: ATCC 393.

Lactobacillus rhamnosus: Agricultural Research Service Culture Collection (NRRL); Address: 1815 N. University Street Peoria, Ill. 61604 USA; Accession number: NRRL B-445.

Lactobacillus delbrueckii: American Type Culture Collection (ATCC); Address: 10801 University Boulevard Manassas, Va. 20110 USA; Accession number: ATCC 9649.

Lactococcus lactis: American Type Culture Collection (ATCC); Address: 10801 University Boulevard Manassas, Va. 20110 USA; Accession number: ATCC 19435.

Bacillus coagulans: American Type Culture Collection (ATCC); Address: 10801 University Boulevard Manassas, Va. 20110 USA; Accession number: ATCC 7050.

Lactobacillus brevis: American Type Culture Collection (ATCC); Address: 10801 University Boulevard Manassas, Va. 20110 USA; Accession number: ATCC 14869.

Sporolactobacillus inulinus: American Type Culture Collection (ATCC); Address: 10801 University Boulevard Manassas, Va. 20110 USA; Accession number: ATCC 15538.

What is claimed is:

1. A method for producing butyric acid and/or a butyrate, comprising fermenting a saccharide-containing substrate in the presence of a first strain and a second strain, wherein the first strain is a butyric acid bacterium and the second strain is at least one of a homofermentative lactic acid bacterium and a facultative heterofermentative lactic acid bacterium.

2. The method as claimed in claim 1, wherein the first strain is Clostridium sp.

3. The method as claimed in claim 1, wherein the first strain is Clostridium tyrobutyricum.

4. The method as claimed in claim 1, wherein the first strain is at least one of Clostridium tyrobutyricum DSM 27751 and Clostridium tyrobutyricum ATCC 25755.

5. The method as claimed in claim 1, wherein the second strain is as least one of Lactobacillus sp., Lactococcus sp., Sporolactobacillus sp., and Bacillus sp.

6. The method as claimed in claim 1, wherein the second strain is as least one of Lactobacillus casei, Lactobacillus rhamnosus, Lactobacillus delbrueckii, Lactobacillus plantarum, Lactobacillus paracasei, Lactobacillus bulgaricusi, Lactococcus lactis, Bacillus coagulans, and Sporolactobacillus inulinus.

7. The method as claimed in claim 1, wherein the second strain is as least one of Lactobacillus casei, Lactobacillus rhamnosus, Lactobacillus delbrueckii, Lactobacillus plantarum, Lactobacillus paracasei, Lactobacillus bulgaricusi, Lactococcus lactis, and Sporolactobacillus inulinus.

8. The method as claimed in claim 1, wherein the first strain is Clostridium tyrobutyricum DSM 27751, and the second strain is as least one of Lactobacillus casei, Lactobacillus rhamnosus, Lactobacillus delbrueckii, Lactobacillus plantarum, Lactobacillus paracasei, Lactobacillus bulgaricusi, Lactococcus lactis, Bacillus coagulans, and Sporolactobacillus inulinus.

9. The method as claimed in claim 1, wherein the saccharide is at least one of glucose, fructose, lactose, sucrose, molasses, and cellobiose.

10. The method as claimed in claim 2, wherein the saccharide is at least one of glucose, fructose, lactose, sucrose, molasses, and cellobiose.

11. The method as claimed in claim 5, wherein the saccharide is at least one of glucose, fructose, lactose, sucrose, molasses, and cellobiose.

12. The method as claimed in claim 1, wherein the saccharide is at least one of glucose, fructose, lactose, sucrose, and molasses.

13. The method as claimed in claim 2, wherein the saccharide is at least one of glucose, fructose, lactose, sucrose, and molasses.

14. The method as claimed in claim 5, wherein the saccharide is at least one of glucose, fructose, lactose, sucrose, and molasses.

15. The method as claimed in claim 1, wherein the substrate further contains a carbon source, a nitrogen source, and/or a mineral element.

16. The method as claimed in claim 2, wherein the substrate further contains a carbon source, a nitrogen source, and/or a mineral element.

17. The method as claimed in claim 5, wherein the substrate further contains a carbon source, a nitrogen source, and/or a mineral element.

18. The method as claimed in claim 15, wherein the carbon source is at least one of acetic acid and acetate, and the mineral element is at least one of phosphorus, sulfur, potassium, magnesium, iron, and manganese.

19. The method as claimed in claim 16, wherein the carbon source is at least one of acetic acid and acetate, and the mineral element is at least one of phosphorus, sulfur, potassium, magnesium, iron, and manganese.

20. The method as claimed in claim 17, wherein the carbon source is at least one of acetic acid and acetate, and the mineral element is at least one of phosphorus, sulfur, potassium, magnesium, iron, and manganese.
